(19) United States
(12) Patent Application Publication
(10) Pub. No.: US 2010/0272762 A1
(43) Pub. Date: Oct. 28, 2010

(54) Freeze-Dried Coated Molded Article

(75) Inventors: Ralf Malessa, Essen (DE); Claudia Elsinghorst, Billerbeck (DE); Anke Niehues, Ochtrup (DE); Stefan Frahling, Billerbeck (DE); Daniel Rauert, Rosendahl Darfeld (DE)

Correspondence Address: DILWORTH & BARRESE, LLP, 1000 WOODBURY ROAD, SUITE 405, WOODBURY, NY, 11797, US

(73) Assignee: DR. SUWELACK SKIN & HEALTH CARE AG, Billerbeck (DE)

(21) Appl. No.: 12/763,663

(22) Filed: Apr. 20, 2010

(30) Foreign Application Priority Data
Apr. 22, 2009 (EP) 09158477.1

(51) Int. Cl. A61K 31/616; A61K 31/375; A61K 8/67; A61K 8/37; A61K 9/00; A61P 3/02; A61P 29/00; A61P 39/06; A61Q 5/00; A61Q 19/08; B05D 7/00

(52) U.S. Cl. 424/400; 514/474; 514/165; 427/212

(57) ABSTRACT

The invention relates to freeze-dried molded articles comprising at least one or more active substances and optionally one or more scaffold-forming agents, optionally one or more auxiliary substances, as well as a coating comprising at least one film-forming agent. Furthermore, the invention relates to methods for manufacturing these freeze-dried molded articles, the combination of such freeze-dried molded articles in kit-of-parts arrangements together with aqueous solutions, as well as the use of the freeze-dried molded articles and the kit-of-parts combinations for pharmaceutical and cosmetic application.

The invention relates to freeze-dried molded articles comprising at least one or more active substances and optionally one or more scaffold-forming agents, optionally one or more auxiliary substances, as well as a coating comprising at least one film-forming agent. Furthermore, the invention relates to methods for manufacturing these freeze-dried molded articles, the combination of such freeze-dried molded articles in kit-of-parts arrangements together with aqueous solutions, as well as the use of the freeze-dried molded articles and the kit-of-parts combinations for pharmaceutical and cosmetic application.

Freeze drying provides a method that makes it possible to produce rapidly hydrating materials. A highly porous matrix is in this case produced during the freeze-drying process, depending on the water content of the substance to be dried. In this case, ice crystals form from the contained water during the freezing process in the matrix solution to be dried, said ice crystals determining the pore structure after sublimation in the freeze-drying process. This pore structure results in a large inner surface area that is pivotal for the rehydration behavior of the freeze-dried material. Depending on the composition of the material, this generally leads to very short rehydration times. Moreover, the composition of the material is also essential for the mechanical stability of the freeze-dried porous matrices. In particular in the production of freeze-dried, active substance-containing materials or molded articles, as they are known and used widely for pharmaceutical and cosmetic use, the mechanical stability and thus handling of the materials after the freeze-drying process play a central role. In order to be able to form active-substance matrices that are stable with respect to their shape, auxiliary substances that provide the freeze-dried final product a certain mechanical strength usually must be added in addition to the actual active substances. For this purpose, scaffold-forming substances and polymers that are harmless for cosmetic or pharmaceutical areas of use, so-called scaffold-forming agents are used, in particular natural polymers, for example from the group of the hydrocolloids, such as particular those based on collagen or based on plant or animal polysaccharide. Generally, the higher the content of scaffold-forming polymers, the higher the mechanical strength of the freeze-dried product and the lower the hydration rate. High polymer concentrations thus, on the one hand, lead to stable and rugged abrasion-resistant and thus dust-free products with good handling properties, which on the other hand, however, require longer for complete wetting, due to the swelling behavior of the polymers contained therein. Lower concentrations of scaffold-forming agents, however, lead to products that can be wetted within a very short period of time, but do not have a sufficient mechanical stability and strength and thus provide extremely fragile and dusty products.

Thus, the obtainable freeze-dried product so far always represents a compromise between a rapid dissolution and sufficient mechanical stability.

In order to stabilized freeze-dried materials with a low mechanical strength, it is conceivable to apply a continuous stable protective layer on the material. Thus, molded articles can be obtained with a recipe distribution consisting of an inner active substance-containing core with very low or completely non-existent content of structural polymers and an outer polymer layer for stabilizing the unstable active substance-containing core.

In this case, coating molded articles with film-forming or structure-providing polymers for modifying the physical/chemical properties of these molded articles, in particular also for stabilizing and influencing the dissolution behavior of such molded articles, are known in principle from the prior art and are used widely and with a broad spectrum of use in the pharmaceutical field, for example in the technology of table coatings. For example, tablets can be produced with acid-resistant coatings in order to obtain formulations of active substances that are resistant to gastric juice. Coatings that enable a controlled or delayed release of active substances for producing so-called sustained release formulations are also known. Moreover, coatings are generally used in the production of tablets for preventing dust formation, moisture absorption and other destabilizing influences, as well as generally for the mechanical stabilization of the materials.

In this case, production is commonly carried out in a three-stage process. After the production of the tablets, they are provided in a second step with a coating dissolved in a solvent, and in a third step, the solvent is removed, for example by drying or evaporation. In order to be able to use this process for coating or covering, a sufficient stability of the material to be coated is required. In this case, because of the production method, the materials must, on the one hand, be stable with regard to mechanical stress because coating is generally carried out in rotating drums or other moving devices in order to obtain a uniform, homogeneous coating. On the other hand, the material must be stable with regard to the coating agent applied. This is relevant in particular with regard to rapidly soluble materials that are to be provided with a coating. In this case, a special challenge is that the material, which is supposed to be characterized by a rapid solubility, can be stabilized with regard to the coating material in such a way that the rapid solubility with regard to the coating materials does not come into effect in this case, or that it can be delayed in such a way by suitable methods that the material remains stable with regard to its shape until the coating has been completed.

Various readily soluble, freeze-dried, active substance-containing molded articles and methods for their production are known from the prior art. For example, JP 2004-149468, EP 0081912 or also U.S. Pat. No. 4,305,502 describe solid active substance compositions stabilized by scaffold-forming polymers that are characterized by a rapid solubility. In these cases, however, these compositions are directly dried and packaged in the molds or final packagings (e.g. bottles or blisters), which is connected with the low mechanical stability of the molded articles obtainable by these methods. Individual molded articles obtainable in a loose form, and thus correspondingly mechanical stable molded articles, cannot be obtained in this manner. Since the materials directly remain in the final packaging, obviously no uniform coating of the material is provide, nor is it possible due to the low stability.

Solid, rapidly soluble active substance preparations stabilized with scaffold-forming polymers are known, moreover, from DE 69227467 or JP 2003-238693, as well as from WO 04/011537 and WO 05/073296, with the preparations described herein have a sufficiently high stabilizing polymer content in the composition, so that an additional stabilizing coating is obviously not necessary and not described, either. However, the above-described drawback of a comparatively poorer solubility immediately results from the comparatively high content of scaffold-forming agent in the compositions disclosed herein. For example, the preparations according to JP 2003-238693 only dissolve by mechanical action, e.g. by rubbing with the fingers, the compositions of WO 04/011537 and WO 05/073296 merely make reference to a good solubility or dispersability without making separate statements with regard to the dissolution rate. In contrast, the preparations described in DE 69227467 obviously exhibit exceptionally good dissolution rate; however, they are provided with the high mechanical stability by a large content of fillers such as lactose or mannitol (more than 50% by wt.), so that preparations with a particularly high content of active substances cannot be obtainable. Thus, the stabilization of such readily soluble preparations with a high content of active substances and low content of structure-providing auxiliary substances by coating is not the subject matter of these disclosures.

Solid active substance preparations that are stabilized with polymer structure-forming agents and that dissolve when liquid is added, and which moreover are modified in their physical/chemical properties by a covering or coating, are described in U.S. Pat. No. 5,843,347, or also in U.S. Pat. No. 5,578,307, U.S. Pat. No. 5,405,616, EP 0701815 and DE 4201179. However, such coatings are disclosed in these documents which affect the dissolution behavior of these active substance preparations in such an extent that no rapid solubility can be obtained anymore. Rather, the coatings are intended for producing gastric juice-resistant preparations or for providing sustained release preparations, which inevitably entails a significantly reduced solubility, for example, in aqueous or physiological media, such as, for example, saliva in the case of oral application. Thus, the preparations disclosed herein offer no suggestions as to a suitable coating technique which, on the one hand, leads to a stabilization of the solid active substance-containing molded articles or preparations, and which, on the other hand, ensures or retains rapid solubility nevertheless. Moreover, all preparations described herein inevitably contain scaffold-forming agent selected from the group of proteins. Such proteinogenic scaffold-forming agent are pivotal for the stability of the freeze-dried compositions, because the latter, as is known, are subjected to cross linking, the so-called dehydrothermal cross linking. A material which is sufficiently stable in order to provided with the film-coating following the freeze-drying process, i.e. In the dried state, is obtained by this cross-linking. Coating in the frozen state is not described. Moreover, because of the mechanical stability of the freeze-dried pellets required for this method, a sufficiently high content of these scaffold-forming polymers is required and compositions with a content of active substances <50% by wt. cannot be obtained by the method described here. The increase of the content of active substances is accompanied by a loss of mechanical stability of the freeze-dried molded article which makes it impossible, in particular if hydrophilic active substances are used, to coat them subsequently, because they collapse immediately when an attempt is made to coat them.

Furthermore. GB 1206033 describes freeze-dried molded food articles, in particular freeze-dried ice cream, which can be coated with chocolate, which can in principle be called a film-forming agent. However, coating is only carried out also in this case after the step of freeze drying, which can be carried out in molding trays that were optionally provided with a coating of film-forming agent. However, a active substance-containing molded article coated with a film-forming agent, is not disclosed here.

Thus, the above-described methods for coating readily soluble solid active substance compositions are only suitable for such preparations which already have as such sufficient mechanical stability. However, if the intention is to stabilize particularly fragile and, as such, mechanically unstable active substance compositions with a coating in order to achieve an increased mechanical stability, in particular those that have no or only an extremely small content of structure-providing or scaffold-forming polymer auxiliary substances or a particularly high content of active substances, the problem arises, on the one hand, that these inherently mechanically unstable preparations are not suitable for most conventional coating methods, because of the mechanical stresses generated in the process. On the other hand, such rapidly soluble solid preparations, due to their high susceptibility to solvent media, must not react with the coating agent in such a way that the dissolutions occurs already during the stabilization or coating method. Moreover, the coating materials used must be such that the coated molded article remains rapidly soluble also after coating. In particular, this is problematic if the finished freeze-dried molded article is finally to be provided with a coating, as is described in the above-mentioned methods. In this case, coating takes place on the solid, dried form of the preparation in all the methods described above.

If an attempt is now made to stabilize freeze-dried molded articles with the desired properties of high content of active substances, few scaffold-forming auxiliary substances, high dissolution rate and a high or sufficient mechanical stability, by a coating for providing loose individual molded articles, in particular for use as a single-unit dosage form, it becomes clear that hydrophilic coating solutions, such as, for example, those based on glycerin, on the one hand increase hygroscopicity, i.e. susceptibility to moisture, of the finished product, and moreover lead to an unstable coating, which shows itself in particular by films of smearing and abrasion on packaging materials. Coating solutions on an aqueous basis are unsuitable for such freeze-dried preparations for the aforementioned reasons, namely high dissolution rate and thus extremely high susceptibility to aqueous solvents. If hydrophobic coatings are used, e.g. based on oils and fats, such as for example neutral oil, or based on hydrophobic polymers, such as described in JP 54105289, this reduces the dissolution behavior of the active substance-containing molded articles in particular in aqueous media or physiological liquids, such as saliva to the extent that the dissolution rate is reduced to a very large degree. This is disadvantageous in particular if a quick release of active substances and thus a quick availability of the active substances is desired. For these reasons, a coating based on a hydrophilic aqueous coating composition is preferred. However, such a coating has so far not been described for the molded articles desired in this case for the above-mentioned reasons, and is not available for the above-mentioned reasons.

One possibility of obtaining a hydrophilic coating on a rapidly soluble molded article lies in applying the coating onto the frozen molded article prior to freeze drying. The advantage lies in the fact that the ice structure provides the composition with sufficient mechanical stability in order for the molded article to withstand the mechanical stress during the coating method. Because the particularly rapid solubility of the compositions substantially results from the pore structure of the freeze-dried compositions, and since this potential for rapid solubility is not present to this degree in the frozen state, the other problem of premature dissolution or liquid-induced instability of the freeze-dried molded article can be sidestepped by coating the frozen compositions with the coating solvent. The coating solvent is in this case removed together with the solvent of the solutions, usually the water content of the active substance composition, in the subsequent step of freeze drying. Moreover, this increases the efficiency of the method, because compared with methods in which the finished product is subjected to a coating step, the additional process step for the removal of the coating solvent is saved. Moreover, the method of coating the frozen molded articles ensures that moisture-susceptible active substances are protected by the deep temperatures in the frozen molded article, whereas such sensitive active substances in already dried compositions are exposed to the coating solvent for a certain time when they are contacted with it, which can possibly lead to significant losses in activity in active substances that can decompose in moisture.

Moreover, the method of coating frozen molded article and subsequently freeze-drying them results in a high variability with regard to the selection of the recipe of the active substance composition. The scaffold-forming structural polymers can be used in significantly lower concentrations. Ideally, their addition can even be dispensed with completely, so that dried molded articles can be obtained that require no structural polymers in the interior of the molded article, and which are mechanically stabilized only by a thin polymeric film-forming coating layer.

Rapidly soluble solid preparations coated in the frozen state are mentioned in the already cited JP 54105289, with, however, ice pellets being coated therein without them being subjected to a subsequent freeze-drying step. Moreover, a hydrophobic coating is used, which is disadvantageous for application of active substances in aqueous systems for the reasons mentioned. Furthermore, hydrophobic coatings for molded articles that are subsequently to be subjected to a freeze-drying process unsuitable because the hydrophobic coating prevents or limits very much the escape of liquid from the interior of the composition during sublimation, which has a disadvantageous effect on the freeze-drying process.

In DE 10248314 or the corresponding WO 2004/035023, rapidly soluble freeze-dried molded article from mixtures of active substances and scaffold-forming agents for external application are described. The possibility of subjecting these molded articles to a surface coating in the frozen state, prior to a freeze drying process, is mentioned. However, only such coatings for applying a layer of active substances or colors are mentioned, and such coatings that reduce the dissolution rate of the molded articles by cross-linking the alginate-based structural polymers contained in the molded article. In this case, the possibility of mechanically stabilizing the molded articles by means of the coating, in particular by a coating with a film-forming agent, is not disclosed, even more so as such a possibility is provided in a sufficient extent already by the scaffold-forming agents, due to the high contents of scaffold-forming structural polymers contained in the active substance compositions disclosed therein of at least 10% by wt., preferably 15-100% by wt. The possibility of obtaining stable freeze-dried molded articles with high contents of active substances and extremely low contents of scaffold-forming agents with a sufficient mechanical stability by means of a thin coating with film-forming agent is not disclosed in DE 10248314 or the corresponding WO 2004/035023. In particular in view of the hitherto limited possibilities of incorporating a number of important and highly potent active substances for cosmetic and pharmaceutical use that are known to be extremely unstable with regard to external influences such as light, temperature, oxidation or moisture, there is a further central interest in bringing such highly potent and highly unstable active substances, which are in danger of being decomposed, into a form which affords high and long-term stability, accompanied by good storability, optimal and reproducible provision of the content of active substances over the entire storage and administration period, and thus the highest possible safety and efficiency in application.

Apart from an effective stabilization of the active substances, in this context, their provision in an optimally suitable form of administration that is optimally adapted to the respective purpose of application is of particular interest. The choice of the suitable form of application in this case particularly depends on the type and place of application, the target group and its special characteristics, the type and quantity of the dosage of the active substances or their form of application, as well as for example, the physical and biochemical characteristics of the active substances, in particular with regard to their biological availability and their systemic mode of action, which must be taken into consideration in this case.

In particular forms of application for external application as well as oral forms of application are of particular interest for providing such stable highly potent active substances in this case. In this context, in particular such forms of administration are particularly suitable and preferred for such applications, which can be used in aqueous and/or water-containing formulations or environments, and which are rapidly soluble in such aqueous environments. This is of importance in particular in the case of systems of active substances for oral application.

So far, various methods, form of administration and application systems were developed in order to stabilize such unstable, easily decomposing and/or easily degradable substances and keep them available also in the long term in cosmetic and/or pharmaceutical composition for external or also oral application. Methods that must be mentioned in this case particularly include those for encapsulating active substances, e.g. in liposomes, the use of special emulsification techniques or stabilizing solvents, or also the provision of unstable active substances in administration systems that comprise a stable derivative form or active substance precursor as well as an agent for converting the active substance from the precursor.

The drawbacks of these methods include the limited loading density, the complex production, the contact of water-susceptible, water-soluble or water-insoluble active substances with the solvent water during the storage period, as well as the mostly insufficient and inadequately reliable and poorly reproducible release kinetics of the stabilized active substances from the compositions.

Moreover, with regard to highly concentrated active substance administration forms, there is a particular interest in so-called single-dose-unit forms of application, which facilitates a simple and specific dose application for the final user. Single-dose-unit forms of application in this context are understood to be application systems, which in contrast to powders or granules contain the desired and required quantity of active substance per application unit in a single application unit, such as tablets or capsules, without, however, having the drawbacks of poor solubility or lack of suitability for external application.

Thus, such easily soluble single-dose-unit forms of application, which are moisture-stabilized by freeze-drying, for oral and/or external application of unstable active substances are becoming increasingly interesting in the form of larger-format embodiments, in particular if large quantities of active substances are to be administered. As a rule, the special challenge in this respect lies in providing high contents of active substances in a rapidly soluble form, and one that is soluble as completely or residue-free as possible, with as high an active substance content as possible, at as small a proportion of carrier or auxiliary substances in the composition as possible being desirable, because, as was already explained in detail, the residue-free and rapid, complete dissolution of the preparation decreases as the content of carrier or auxiliary substances increases.

Thus, there is the necessity of providing large-format single-dose-unit forms of application that can be dosed well, which have a high active substance load, in particular load of unstable active substances with as low a content of insoluble or swellable carrier substance as possible, and thus complete solubility that is as quick as possible, and the highest possible mechanical stability for cosmetic and pharmaceutical external and oral use.

Thus, the object of the present invention lay in providing a composition in which extremely high quantities of active substances, in particular unstable active substances, could be stabilized for a long period of time, and could be released and applied quickly, efficiently, specifically and highly actively during application, wherein the stabilization of the active substances was preferably to be achieved by freeze drying the active substance compositions. Moreover, the object lay in designing these stable active substance compositions in such a way that they have high mechanical strength and sufficient size in order, in particular, to be capable of being used for cosmetic or pharmaceutical application in the form of so-called single-dose-units or single-dosage applications. In this case, the compositions are supposed to be equally suited for external application as well as for an oral or peroral application. Furthermore, it was an object of the invention to find a possibility for this mechanical stabilization which did not have an adverse effect on the dissolution rate of the freeze-dried compositions and which ensures a high dissolution rate of the freeze-dried final product, in particular by reducing as far as possible the content of stabilized structure-providing scaffold-forming agents, ideally avoiding them completely.

Surprisingly, it was found that, based on DE 10248314, such stable, large-format, active substance-loaded molded articles could be produced that contain quantities of active substances, which at ≧50% by wt. content of active substances and <10% by wt. content of scaffold-forming agents, lie outside of the quantities disclosed in DE 10248314, by compensating the accompanying problems of mechanical stability by means of coating the molded articles. Surprisingly, in particular unstable active substances could be introduced in large quantities without the required stability being achieved by large contents of scaffold-forming agents, but the dissolution rate being reduced.

By selecting a suitable film-forming hydrophilic coating and applying this coating onto the frozen molded article, and subsequent freeze drying of the coated, frozen molded article, it was possible, surprisingly, to obtain a freeze-dried, mechanically stable, highly concentrated active substance molded article, which, due to the extremely low content in scaffold-forming agents according to the invention moreover could again be significantly improved with regard to its dissolution behavior as compared with the systems already known, such as those described in DE 10248314.

Neither DE 10248314 nor any of the other documents discussed herein disclose solid freeze-dried molded articles with such a high content of active substances and with a coating of a film-forming agent which has such good properties with regard to mechanical stability, dissolution behavior and size for the application in the cosmetic and pharmaceutical single-dose application.

Thus, the invention provides freeze-dried molded articles comprising one or more active substances and optionally one or more scaffold-forming agents, optionally one or more auxiliary substances, as well as a coating comprising at least one film-forming agent.

Furthermore, the invention also provides a method for manufacturing such freeze-dried molded articles, the combination of such freeze-dried molded articles in kit-of-parts arrangements together with aqueous solutions, as well as the use of the freeze-dried molded articles and the kit-of-parts combinations for pharmaceutical and cosmetic application.

A molded article within the sense of the invention is understood to be a geometric body of a regular shape, for example, in particular, spheres, cuboids, pyramids, stars, but also molded articles replicating natural shapes, such as those in the form of animals, such as marine animals, such as starfishes, seafood, such as mussels, etc, plants and parts of plants, such as leaves etc. According to the method for producing the molded articles used according to the invention described below, all of these shapes are obtainable. Uniform spherical shapes are preferred according to the invention, such as, in particular, a spherical geometry, because, with regard in the case of coating with the film-forming coating, it has shown itself to be particularly advantageous with regard to homogeneous coating, and because it can be processed particularly well to form mechanically stable molded articles because of the particularly favorable ratio of surface area/volume. The sublimation distance through the already dry product is symmetric and small to all sides in spherical or ball-shaped molded articles, which facilitates vapor transport through the already dry material within the context of the freeze-drying process.

In one aspect of the invention, a plurality of the molded articles mentioned are included in a container. This may also include mixtures of molded articles with different geometries or different sizes. The molded articles may be packaged individually, preferably, however, in particular in cosmetic application, a plurality of the molded articles lies in contact with each other next to each other in a container.

The volumes of the molded articles used are not limited as such by the method of producing them. Expediently, the volumes are preferably about 0.1 cm³, preferably 0.3 cm³, more preferably at least about 0.5 cm³, still more preferably at least about 0.6 cm³. The upper limit of the volumes used is expediently about 6 cm³, preferably about 5 cm³, more preferably about 4 cm³. Among other things, the size of the molded articles is determined by the desired form of application or the location of the external application of the molded articles. Thus, in external cosmetic or pharmaceutical use, the application to larger body surfaces or on the hair (e.g. direct application of the moistened molded articles on the back etc., or the use as a bath preparation) makes the use of larger molded articles possible, whereas smaller molded articles are preferred during use on smaller areas of the body (e.g. the cheek, etc.).

The size can also be adapted in the production of molded articles for oral application. For example, it is conceivable to adapt the size of the molded articles to the relevant target group, wherein it is conceivable that older users are offered larger molded articles that can be handled more easily, and to offer, for example, younger users and children such molded articles that have an adapted relationship to their body size and the compliance in application to be expected because of their age.

The diameter of a molded article (maximum distance between two points in a molded article of any geometry) expediently is at least about 3 mm, preferably at least about 5 mm, more preferably at least about 7 mm, still more preferably at least about 8 mm, up to, expediently, 60 mm, preferably about 50 mm, more preferably about 40 mm, still more preferably about 20 mm.

For the aforementioned reasons, a particularly preferred molded article has a substantially spherical geometry, with the diameter of the sphere being between 3 to 30 mm, preferably between 5 and 20 mm, more preferably between 7 and 15 mm, still more preferably between 8 and 14 mm. Molded articles in the shape of a sphere with a diameter of at least 6 mm are particularly preferred.

The freeze-dried molded articles according to the invention comprise at least one or more active substances, preferably at least one active substance in an amount of ≧50% by wt., based on the coated freeze-dried total composition. Active substances in particular include cosmetic or therapeutic or pharmaceutical active substances suitable for external use as well as for oral or peroral application. Preferably, the molded article used according to the invention comprises at least one cosmetic and/or pharmaceutical active substance. Accordingly, the freeze-dried molded articles according to the invention preferably are preferably cosmetic or pharmaceutical products.

Cosmetic molded articles or molded articles produced using cosmetic active substances within the sense of the invention are substantially products within the sense of the German Food and Feed Code (LFGB), i.e. substances or preparations from substances that are intended for external application on humans for cleaning, care or influencing appearance or body odor, or for conveying olfactory impressions, unless they are primarily intended for alleviating or eliminating disease, disorders, bodily defects or pathological complaints. In this sense, the cosmetic molded articles used according to the invention are, for example, bathing preparations, skin washing and cleansing products, skin care products, in particular facial skin care products, eye cosmetics, lip care products, nail care products, foot care products, hair care products, in particular hair washing products, hair conditioning products, hair softening rinse etc., light protection products, suntan products and skin lightening products, depigmentation products, deodorants, antihydrotics, depilatory products, insect repellents etc. or a combination of such products.

Examples of cosmetically, or optionally, for example, dermatologically therapeutically effective substances can be: anti-acne products, antimicrobial products, antitranspiration products, astringent products, deodorizing products, depilatory products, conditioning products for the skin, skin-smoothing products, products for increasing skin hydration, such as glycerin or urea, sun blockers, keratolytic products, free-radical scavengers for free radicals, antiseborrhoeic products, anti dandruff products, antiseptic active substances, active substances for treating signs of the aging of the skin and/or products modulating the differentiation and/or proliferation and/or pigmentation of the skin, vitamins, such as vitamin C (ascorbic acid) and its derivatives, active ingredients with a stimulating side effect, such as alpha-hydroxy acids, β-hydroxy acids, alpha-keto acids, β-keto acids, retinoids (retinol, retinal, retinic acids), anthralins (dioxanthranol), anthranoids, peroxides (in particular benzoyl peroxide), minoxidil, lithium salts, antimetabolites, vitamin D and its derivatives; catechins, flavonoids, ceramides, polyunsaturated fatty acids, essential fatty acids (e.g. gamma-linolenic acid), proteinogenic active substances with a molecular weight of less than 1000 kDa, such as enzymes, coenzymes, peptides, enzyme inhibitors, hydrating agents, skin-soothing agents, detergents or foam-forming agents, and inorganic or synthetic matting fillers, or decorative substances such as pigments or colorants and particles for foundations, make-up formulations, and other products for cosmetic beautification and coloring of the eyes, lips and face, as well as abrasive products.

Moreover, plant substance extracts or extracts obtained therefrom or individual substances can be mentioned. Generally, the plant active substance extract is regularly selected from the group consisting of solid plant extracts, liquid plant extracts, hydrophilic plant extracts, lipophilic plant extracts, individual plant ingredients and their mixtures, such as flavonoids and its aglycones: rutin, quercitin, diosmin, hyperoside, (neo)hesperidine, hesperitine, Ginkgo biloba (e.g. ginkgo flavone glycosides), Crataegus extract (e.g. oligomer procyanidines), buckwheat (e.g. rutin), Sophora japonica (e.g. rutin), birch leaves (e.g. quercitin glycosides, hyperoside and rutin), elderberry blossoms (e.g. rutin), lime blossom (e.g. essential oil with quercitin and farnesol), oil of St. John's wort (e.g. olive oil extract), calendula, arnica, (e.g. oily extracts of the blossoms with essential oil, polar extracts with flavonoids), melissa (e.g. flavones, essential oils), immunostimulants: Echinacea purpurea (e.g. alcoholic extracts, fresh plant juice, press-juice), Eleutherokokkus senticosus; alkaloids: Rauwolfia (e.g. Prajmalin), Vince (e.g. vincamin); other phytopharmacons: Aloe, horse chestnut (e.g. aescine), garlic (e.g. garlic oil), pineapple (e.g. bromelaines) ginseng (e.g. ginsenosides), Silybum marianum fruits (e.g. extract standardized to silymarin), Butcher's broom wort (e.g. ruscogenin), valerian (e.g. valepotriates, Tot. Valerianae), Kava-Kava (e.g. kavalactone), hop (e.g. hop bitter substances), Extr. passiflorae, gentian (e.g. ethanolic extract), anthraquinone-containing drug extracts, e.g. aloin-containing aloe vera juice, pollen extract, algae extract, liquorice extracts, palm extracts, galphimia (e.g. mother tincture), mistletoe (e.g. aqueous-ethanolic extract, phytosterols (e.g. beta-sitosterin), common mulleins (e.g. aqueous-alcoholic extract), drosera (e.g. liqueur-wine extract), sea-buckthorn fruits (e.g. juice obtained therefrom or sea-buckthorn oil), marshmallow root, primrose-root extract, fresh plant extracts from mallow, comfrey, ivy, horsetail, yarrow, ribwort (e.g. pressed juice), stinging nettle, celandine, parsley; plant extracts from Norolaena lobata, Tagetes lucida, Teeoma slams, Momordica charantia and aloe-vera extracts.

Preferred cosmetic and pharmaceutical active substances include those that have a high instability with regard to decomposition or degradation, in particular as caused by the addition of moisture, as well as those active substances which generate an acid pH value in an aqueous solution due to acid groups in the active substance, so-called acid active substances, which so far could not be satisfactorily transferred into stable freeze-dried forms even by freeze-drying processes, or only in very small concentrations or with very high cost expenditure by a process at very low temperatures with very long drying times.

A particularly preferred active substance from the group of these unstable, acid active substances, which is widely used in cosmetics, is ascorbic acid or vitamin C and its derivatives, or also vitamin A and its derivatives.

Derivatives of ascorbic acid include, in particular, glycosides such as ascorbyl glucoside or esters of ascorbic acid, such as sodium or magnesium ascorbyl phosphate or ascorbyl palmitate and stearate, as well as, for example, L-ascorbic acid phosphate esters, alkaline metal salts, such as sodium and potassium salts of L-ascorbic acid phosphate esters; earth-alkaline metal salts such as magnesium and calcium salts of L-ascorbic acid phosphate esters; trivalent metal salts such as aluminum salts of L-ascorbic acid phosphate esters; alkaline metal salts, such as sodium and potassium salts of L-ascorbic acid sulfate esters; earth-alkaline metal salts such as magnesium and calcium salts of L-ascorbic acid sulfate esters; trivalent metal salts such as aluminum salts of L-ascorbic acid sulfate esters; alkaline metal salts, such as sodium and potassium salts of L-ascorbic acid esters; earth-alkaline metal salts such as magnesium and calcium salts of L-ascorbic acid esters; and trivalent metal salts such as aluminum salts of L-ascorbic acid esters.

In contrast to the above described molded articles, which are substantially used in cosmetics, the therapeutically used molded articles (medicaments) are such molded articles containing at least one pharmaceutical or therapeutic, in particular dermatologically active substance, and which, within the meaning of the “Arzneimittelgesetz” (German Medical Preparations Act), are intended to cure, to ease or to prevent diseases, illnesses, bodily damage or pathological complaints. Such agents or active substances are intended both for external use as well as oral or peroral application.

Active substances for external use are in particular skin-active, but also transdermal active substances. They include, for example:

agents for the treatment of skin diseases, externally applicable analgesics, e.g. dextropropoxyphene, pentazocine, pethidine, buprenorphine; antirheumatics/antiphlogistics (NSAR), e.g. indomethacin, diclofenac, naproxen, ketoprofen, ibuprofen, flurbiprofen, salicylic acid and salicylic-acid derivatives such as acetylsalicylic acid, oxicams; steroid hormones, e.g. betamethasone, dexamethosone, methylprednisolone, ethynyl estradiol, medroergotamine, dihydroergotoxine; gout remedies, e.g. benzbromarone, allopurinol; external dermatological agents, including antibacterial agents, such as e.g. colloidal silver or silver salts, antimycotics, antiviral active substances, anti-inflammatory active substances, antipruritic active substances, anaesthetizing active substances, e.g. benzocaine, corticoids, anti-acne agents, antiparasitic active substances; externally applicable hormones; venous therapeutic agents; immunosuppressives etc., all for external application.

Preferred therapeutic products for external use are analgesics, e.g. immunosuppressives, hormones, products for the treatment of skin diseases such as neurodermitis, atopical dermatitis, acne, rosacea etc and anti-herpes products.

Therapeutic active substances for oral or peroral application can be selected from the group of antihistamines, antibiotics, peptide drugs, antimycotics, bronchial therapeutics such as antiasthmatics, antitussives, mucolytics, etc., antidiabetics, such as glibenclamide, hormones, steroid hormones, such as dexamethasone, cardiac glycosides such as digitoxin, heart and circulation therapeutics such as, e.g., beta blockers, antiarrhythmics, antihypertonics, calcium antagonists etc., psychopharmaceutical agents and antidepressants, such as tricyclic antidepressants (NSMRI), serotonin-specific reuptake inhibitors (SSRI), norepinephrine reuptake inhibitors (NRI), serotonin and norepinephrine reuptake inhibitors (SNRI), monoamine oxidase inhibitors (MAO inhibitors), etc., neuroleptics, anticonvulsives or antiepileptics, hypnotics, sedatives, anaesthetics, gastro-intestinal therapeutics, lipid-lowering drugs, analgesics, such as anti-migraine agents, paracetamol, salicylic acid and its derivatives such as acetylsalicylic acid, diclofenac, ibuprofen, ketoprofen, naproxen etc, antiphlogistics, vasodilators, diuretics, antipodagric agents, cytostatic agents, muscle relaxants, plant extracts, provitamins, such as beta carotene, vitamins such as vitamin C (ascorbic acid), A, B, E etc., silica, minerals and trace elements such as, e.g., potassium, magnesium, calcium, selenium, iodine, etc., dietary supplements and food supplements etc, all for oral and peroral application.

A particularly preferred pharmaceutical active substance which is used both for external as well as oral or peroral application and selected from the group of unstable, acid substances is salicylic acid and its derivatives, such as acetylsalicylic acid (ASS). Other preferred unstable, acid and freezing-point lowering active substances are clofibric acid, ibuprofen, gemfibrozil, fenoprofen, naproxen, ketoprofen, indomethacin, bezafibrate, tolfenamine acid, diclofenac, meclofenamine acid, paracetamol, acitretine, acrivastine, azelaic acid, cromolyn, ethacrynic acid, furosemide, penicillin and derivatives thereof, risedronic acid and derivatives thereof, lipoic acid and ursodiol.

The freeze-dried molded articles according to the invention preferably have a content of active substances of ≧50% by wt., preferably ≧75 by wt., more preferably ≧80% by wt., still more preferably ≧90% by wt., in each case based on the total composition of the freeze-dried, coated molded articles.

These are in particular such active substances which are selected from the group of the acid active substances, that is, active substances that generate an acid pH value in an aqueous solution due to acid groups in the active substance. Such acid active substances are in particular active substances whose 1% by wt. solution or suspension in water, at 20° C., has a pH value <7, or such active substances that have a pKa value, at 25° C., of ≦7.

The pKa value in this case denotes the negative decadic logarithm of the acid constant Ks. The acid constant is a material constant and furnishes information on the extent a substance (HA) reacts in an equilibrium reaction with water under protolysis.

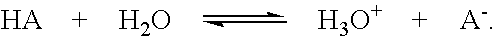

In this case, HA represents a Brønsted acid (after Brønsted), which can donate a proton H⁺ to a solvent such as water, leaving behind an anion A⁻. More generally, the Brønsted definition also applies to non-aqueous systems, in this case, the following applies for any protonable solvent Y:

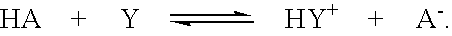

The acid constant Ks in this case denotes the equilibrium constant of this reaction and is a measure for the strength of an acid. The stronger the acid, the more the reaction is shifted towards the right side. This shows that, the smaller the pKs value, the stronger the acid.

Determination of the pKs value is carried out by measuring pH in a so-called half-titration. In the process, a solution of the acid of known concentration is provided and the pH value is measured, for example, by means of a pH measuring probe. Then, the acid is partially neutralized with a standard solution of a base of the same valence as the provided acid. In the process, exactly half of the substance quantity of the provided acid is added. The pH value is now determined again. The following applies:

$${pK}_{s} = - {lgK}_{s} = - lg \frac{c [ {H}^{+} ] \cdot c [ {A}^{-} ]}{c [ HA ]}$$

Because after the addition of half of the substance quantity c[A⁻]=c[HA], pKs=pH applies for the so-called half-titration point.

Such acid active substances have a pH value-dependent high dissociation tendency, which is why the active substance is present in neutral to alkaline pH value ranges in a dissociated form and thus, in a high ion concentration. Such an increased ion concentration then results in a freezing-point lowering action with the aforementioned disadvantageous effects on the freeze-drying process. Compositions containing, in particular, high quantities of freezing-point lowering substances. Due to the freezing-point lowering effect of such dissociated active substances, large ice crystals with a large content of unfreezable water with high concentrations of active substances generally form in the frozen molded article, which lead to a partial structural collapse of the freeze-dried final product, the so-called thawing of the molded article, which is why such acid active substances could not be satisfactorily transferred into stable freeze-dried forms by freeze-drying processes, or only in very small concentrations or with very high cost expenditure by a process at very low temperatures with very long drying times.

In a preferred embodiment, the freeze-dried molded articles according to the invention, based on the total composition of the freeze-dried coated molded article, contain ≧50% by wt. of an active substance from the group of ascorbic acid and its derivatives.

In another preferred embodiment, the freeze-dried molded articles according to the invention, based on the total composition of the freeze-dried molded article, contain ≧50% by wt. of an active substance from the group of salicylic acid and its derivatives, preferably from the group of acetylsalicylic acid and its derivatives.

The molded articles according to the invention preferably contain only very small amounts of up to maximally 25% by wt., preferably up to maximally 10% by wt., of one or more scaffold-forming agent, based on the total composition of the freeze-dried coated molded article. Such scaffold-forming agents generally are so-called hydrocolloids, i.e. (partially) water-soluble or water swellable, natural or synthetic polymers that form gels or viscous solutions in aqueous systems. Expediently, the scaffold-forming agent are selected from polysaccharides, mucopolysaccharides, animal polyaminosaccharides such as chitin or its derivatives, in particular chitosan, or from the glucosaminoglycans as well as the synthetic polymers. Preferably, the scaffold-forming agent is selected from the group of polysaccharides. Polysaccharides include, for example, homoglycans or heteroglycans, such as, for instance, alginates, in particular sodium alginate, carrageenan, pectins, gum tragacanth, guar gum, pullulan, trahalose, carob gum, agar-agar, gum arabic, xanthan gum, natural and modified starches, such as cationically modified starch derivatives, dextrans, dextrin, maltodextrins, glucans, such as β-1,3-glucan or β-1,4-glucan, such as cellulose, mucopolysaccharides, such as hyaluronic acid etc, as well as animal polyaminosaccharides, such as chitin or its derivatives, such as in particular chitosan. Synthetic polymers include, for example: cellulose ether, polyvinyl alcohol, polyvinyl pyrrolidone, synthetic cellulose derivatives, such as methylcellulose, carboxycellulose, carboxymethylcellulose, cationized carboxymethylcellulose, cellulose ester, cellulose ether such as hydroxypropylcellulose, polyacrylic acid, polymethacrylic acid, poly(methylmethacrylate) (PMMA), polymethacrylate (PMA), polyethylen glycols, etc. Mixtures of several scaffold-forming agents can also be used. According to the invention, alginates, such as, in particular, sodium alginate, are particularly preferred. Hydrophilic and readily water-soluble scaffold-forming agents are preferred, in particular calcium-free sodium alginates (sodium alginates with a calcium content of <3% by wt., more preferably <2% by wt., still more preferred <1.5% by wt.), celluloses such as carboxymethylcellulose, hyaluronic acid as well as chitosan or cationically modified starch or cationically modified carboxymethylcellulose.

In a preferred embodiment, the freeze-dried molded articles according to the invention comprise at least one scaffold-forming agent selected from the group of the cationic scaffold-forming agents. Generally, these are understood to be those scaffold-forming agents that, under physiological environmental conditions (room temperature, neutral pH value range, aqueous environment) have more positive charges than negative charges on their surface.

In particular, cationically modified polymers comprise those in which at least one side group of the polymer skeleton is substituted by cationic groups. According to the invention, such cationically modified polymers are particularly preferred that have a degree of substitution (Sga) ≧1%. In this case, the degree of substitution, depending on the type of cationic group in the modified polymer, can be determined in accordance with the respectively suitable standard testing methods known to the person skilled in the art.

Generally, the term cationic polymers or scaffold-forming agent comprises in particular modified chitin derivatives, such as, in particular, chitosan, but also other chemically modified biopolymers, such as cationized celluloses, e.g. carboxymethylcellulose or cationized starches.

Cationic biopolymers based on polysaccharides, such as celluloses or starches in this case comprise those in which some hydroxy groups in the polymer side chains are etherified with cationic groups or groups which, in an acid medium, can be converted into cationic groups by protonation. These substituents can consist, for example, of tertiary amino groups or quaternary ammonium salts, or also of sulfonium groups or phosphonium groups.

In particular if active substances from the group of acid active substances, such as, in particular ascorbic acid and its derivatives or acetylsalicylic acid and its derivatives, are used, the use of readily soluble cationic scaffold-forming agents, such as chitosan or cationized starch derivatives or cationized cellulose derivatives (e.g. carboxymethylcellulose), optionally also mixed with others of the above-mentioned scaffold-forming agents, is particularly preferred.

In a preferred embodiment, the use of scaffold-forming agents from the group of proteins is excepted.

The use of such hydrophilic, readily water-soluble scaffold-forming agents is preferred, on the one hand, because of the production process, on the other hand, the use of such hydrophilic scaffold-forming agents least to a ready solubility of the formulation, e.g. in the mouth, by means of saliva or, if water or aqueous solutions are added, to a high decomposition or dissolution rate, and thus, to an easy dispersability on the skin. In particular the use of readily soluble scaffold-forming agents in the preferred low contents of ≦10% by wt, based on the total composition of the freeze-dried coated molded article, can lead to a higher dissolution rate of the molded articles according to the invention. Particularly preferred embodiments require no addition at all of such scaffold-forming structural polymers in the active substance composition.

For clarification, a distinction must be made between those polymeric scaffold-forming agents that are present in a homogeneous distribution with the active substance-containing composition, i.e. in the inner core area of the freeze-dried molded articles, and those polymers that form the outer coating. One above-mentioned particularly preferred embodiment that requires no scaffold-forming structural polymers, according to the invention nevertheless comprises an outer coating of a film-forming polymer as is defined below. The lack of scaffold-forming structural polymers in such preferred embodiments in this case relates to the active substance composition forming the inner area of the molded article.

The use of small quantities of scaffold-forming substances in the active-substance molded articles according to the invention as compared to, for example, pure, dried, additive-free active substances was an absolute necessity so far, in order to be able to immediately provide the active substance in a suitable, mechanically stable form of administration, which plays a role in particular in external use with regard to applicability and handling. On the other hand, the contents of scaffold-forming agents are decisive in achieving a sufficient stability of the active-substance molded articles.

The polysaccharides which, according to the invention, are preferably used as scaffold-forming agents, expediently have average molar masses of about 10³ to about 10⁸, preferably about 10⁴ to 10⁷.

The freeze-dried molded articles according to the invention are preferably characterized in that they comprise a content of scaffold-forming agents of ≦25% by wt., preferably ≦10 by wt., more preferably ≦5 by wt., in each case based on the total composition of the freeze-dried coated molded article.

The scaffold-forming agent are skin- and mucosa-compatible, and have no toxicological potential; they do not cause irritating effects or other incompatibility reactions, neither during external nor in the oral or peroral application. They are pharmacologically harmless and thus optimally suitable as carrier materials for the cosmetic and pharmaceutical external and oral or peroral uses according to the invention.

These scaffold-forming agents, in particular the polysaccharides, can also have certain therapeutic effects. Thus, the scaffold-forming agent (sodium) alginate, which is preferably used, to a certain extent has an antiviral action, however, it is not an active substances within the sense of the invention.

The molded articles according to the invention furthermore optionally contain one or more auxiliary substances. Auxiliary substances include: fatty substances, such as mineral oils, such as paraffin oils or Vaseline oils, silicone oils, vegetable oils such as coconut oil, sweet almond oil, apricot oil, corn oil, jojoba oil, olive oil, avocado oil, sesame oil, palm oil, eucalyptus oil, rosemary oil, lavender oil, pine oil, thyme oil, mint oil, cardamom oil, orange-blossom oil, soybean oil, bran oil, rice oil, rapeseed oil and castor oil, wheat-germ oil and vitamin E isolated therefrom, evening-primrose oil, vegetable lecithins (e.g. soybean lecithin), sphingolipids/ceramides isolated from plants, animal oils or fats, such as tallow, lanolin, butyric oil, fatty-acid esters, esters of fatty alcohols such as triglycerides, and waxes with a melting point corresponding to skin temperature (animal waxes such as beeswax, carnauba wax and candelilla wax, mineral waxes, such as microcrystalline waxes, and synthetic waxes, such as polyethylene waxes or silicone waxes), as well as all oils that are suitable for cosmetic purposes (so-called cosmetic oils), such as, for example, those mentioned in the CFTA treatise entitled Cosmetic Ingredient Handbook, 1st edition, 1988, The Cosmetic, Toiletry and Fragrance Association, Inc., Washington, pH-adjusting agents, such as buffering substances, surfactants in addition to the above-mentioned washing tensides, such as dispersants, emulsifiers etc, fillers, stabilizers, cosolvents, pharmaceutically and cosmetically commonly used or other colorants and pigments, in particular those that are used primarily for the color design of the molded articles and not for application and color design on the human body, such as those pigments and colorants as those decorative colorants, preserving agents, softening agents, lubricants listed in the group of active agents.

A particularly preferred auxiliary substance is neutral oil (caprylic/capric acid triglycerides).

Other auxiliary substances preferred according to the invention are mannitol, as well as from the group of the pH-adjusting agents, hydrochloric acid and sodium hydroxide solution.

Auxiliary substances can be added to the freeze-dried molded articles according to the invention in quantities of up to 50% by wt. based on the total composition.

The molded articles according to the invention preferably have a total content of active and auxiliary substances of ≧50% by wt., preferably ≧75% by wt., more preferably ≧80% by wt., still more preferably ≧90% by wt., in each case based on the total composition of the freeze-dried, coated molded articles.

The weight ratio of active substances to auxiliary substances in the freeze-dried coated molded articles is preferably approx. 10:1 to 100:1, more preferably approx. 20:1 to 50:1, with a weight ratio of 20:1 being particularly preferred, and wherein the total amount of the active substances in % by wt. is put into relationship with the total amount of the auxiliary substances in % by wt.

The molded articles according to the invention serve for external cosmetic and pharmaceutical and oral or peroral use in humans and animals. External use is carried out such that the molded article according to the invention is moistened or dissolved with water or an aqueous solution, which can optionally contain one or more active substances and/or one or more auxiliary substances, additionally. Depending on the liquid quantity and the solubility of the material of the molded article used, the molded article can be dissolved completely while forming a solution, or decompose while forming a gel, in order then to by applied onto the hair or skin.

Preferably, aqueous solutions are used for dissolution which in addition can contain polyalcohols, as well as those that have a low viscosity (a viscosity <50 mPas) and no or a merely low oil content (<10% by wt based on the total composition of the aqueous solution). Moreover, such activator solutions are preferred that are free from earth-alkaline ions, such as in particular calcium and/or magnesium ions (less than 1% by wt of calcium and/or magnesium ions based on the total composition of the aqueous solution contained), as well as those having a pH value of between about pH 5 to 7.

The solution of the molded article according to the invention in an amount of water suitable for a bathing application is also contained, according to the invention, in the external use. However, use is preferably carried out such that the molded articles are moistened with a small quantity of approximately 0.5 to 5.0 ml, or of approximately 10 to 100 times the own weight of the molded article, water or a solution of active substances and/or auxiliary substances while forming a solution or a gel, directly on the skin or the hair or in a suitable container, decomposing there completely within ≦30 seconds and dissolving preferably completely and without leaving any residue. Preferably, dissolution in this case occurs without any mechanical influence, e.g. by stirring, rubbing, squashing or massaging, or only by mechanical stresses so slight as to be sufficient for causing the stabilizing coating shell to break open. Preferably, the mechanical stress caused by applying an activator solution and the hydrostatic pressure of the activator liquid connected therewith is already sufficient for this purpose.

The present invention also relates to a combination comprising at least one of the molded articles used according to the invention, as well as at least one aqueous solution optionally containing one or more active substances and/or at least one or more auxiliary substances (a so-called activator solution), in a combined spatial arrangement (application package, set, kit-of-parts etc.). The solutions of active substances can be, for example, solutions of highly volatile active and/or auxiliary substances, which should not or cannot be introduced into the molded article by freeze-drying because of the production process, such as certain parts of essential oils, perfumes, etc. Those active and/or auxiliary substances can also be contained which have a moisturizing effect which is desired and preferred in particular with regard to the external use on the skin, and which due to this moisturizing effect or due to hygroscopic tendencies cannot be incorporated into the freeze-dried molded article according to the invention, because thereby, the stability of the moisture-labile active substances cannot be maintained any longer. One example of such a moisturizing, but hygroscopically acting substance is, for example, glycerin.

The configuration of such kit-of-parts combinations of, on the one hand, molded articles according to the invention and the active substance solution, on the other hand, can provide that the two components are removed separately from the kit-of-parts arrangement and are combined and dissolved outside of it for further use. It is also conceivable, however, that a combination of the two components is carried out within the kit-of-parts packaging itself, and that the dissolved composition is then directly supplied from it to the further cosmetic or pharmaceutical external, oral and/or peroral use. Preferably, this can be done directly by the end user.

The molded articles according to the invention preferably contain ≧50% by wt., preferably ≧75% by wt., more preferably ≧80% by wt., still more preferably ≧90% by wt., of one or more active substances, in each case based on the total composition of the freeze-dried, coated molded articles. Thus, the quantity of active substances specified relates to a freeze-dried molded article including the coating with the film-forming agent.

Moisture-labile and/or acid active substances are particularly preferred, such as, in particular, ascorbic acid (vitamin C) and its derivatives, and/or salicylic acid and its derivatives, such as acetylsalicylic acid (ASS).

The content of active substances in the dry total composition can be determined by suitable recognized analysis methods, such as according to DIN, Pharm. Eur., Amtliche Sammlung von Untersuchungsverfahren (ASU, Official Collection of Testing Methods), DAB, USP, etc. The choice of a suitable method is of course dependent upon the kind of active substance. In particular the particularly preferred active substances such as ascorbic acid (vitamin C) and its derivatives, and/or salicylic acid and its derivatives, such as acetylsalicylic acid (ASS) can be analyzed by high performance liquid chromatography methods (HPLC). HPLC methods for quantitative determination of vitamin C and acetylsalicylic acid can be taken from the official monographs “Aspirin Tablets” and “Ascorbic acid Injections” from USP 31, NF 26 Volume 2, 2008, optionally with adaptation of the sample preparation.

Depending on the amount present and the type of the active substances present and/or possible additional auxiliary substances, the molded article according to the invention contains ≦25% by wt. of a scaffold-forming agent, or ≦10% by wt. of a scaffold-forming agent, based on the total weight of the coated freeze-dried molded article, with a content of ≦7% by wt. being more preferred, a content of ≦5% by wt. of the scaffold-forming agent being even more preferred, with polysaccharides, such as sodium alginate or chitosan or carboxymethylcellulose being particularly preferred.

The integral content of scaffold-forming agents in the dry total composition can in this case be determined by hydrolysis of the polymer chains present with a subsequent quantitative chromatographic detection of the individual monomer components. In the event this method cannot be used because of a special combination of different scaffold-forming agents and special active and auxiliary substances, the quantitative polymer content can be determined mathematically via the difference between the total weight and the quantitatively determinable auxiliary and active substances and water. The quantitative methods for determining the individual components of the recipe are borrowed from the official collections of methods already mentioned above.

The molded articles can contain up to about 20% by wt. of one or more auxiliary substances, preferably ≦15% by wt., more preferably ≦10% by wt.

Preferred auxiliary substances are in this case selected from the group of fatty substances and oils, in particular from the group of cosmetic fats and oils. A particularly preferred auxiliary substance is neutral oil (caprylic/capric acid triglycerides), jojoba oil as well as squalane.

The molded articles according to the invention comprise a covering or a coating with at least one film-forming agent. Film-forming agents in particular include synthetic and natural polymers and copolymers, such as, for example, those structural polymers listed under the hydrocolloids. Moreover, semi-synthetic cellulose derivatives, such as hydroxypropyl methylcellulose HPMC, polyvinyl acetate (PVA), polyvinyl pyrrolidone (PVP), shellac, polyvinyl acetate phthalate (PVAP), synthetic acrylic polymers such as, e.g. methacrylate (Eudragit), zein etc.

In addition, polymers can be used which are mentioned in Seitz, J. A. Aqueous Film Coating [Encyclopedia of pharmaceutical technology, J. Swarbrick and J. C. Boylan; Marcel Dekker, New York (1988) 1; 337-349] and Cole, G. C. Introduction and overview of pharmaceutical coating, in Pharmaceutical Coating Technology, G. Cole, J. Hogan and M. Aulton; Taylor and Francis LTD., London (1995), 1-5., as well as in Aulton M., Mechanical Properties of film coats, Pharmaceutical coating technology, G. Cole et. al., Taylor and Francis, London (1995), 280-362.

According to the invention, the film-forming agent is preferably selected from the group of hydrophilic polymers, preferably from the group of synthetic polymers, particularly preferably from the group of vinylpyrrolidone/vinylacetate copolymers (e.g. Kollidon VA64®).

The proportion of the coating with the film-forming agent in the freeze-dried total composition including the coating preferably is ≦10% by wt, preferably, the coating represents ≦7% by wt., still more preferably ≦5% by wt. of the coated freeze-dried total composition.

Optionally, the molded articles also contain water residues. Since the active substances contained in the molded articles, according to the invention, are to be protected particularly against moisture as well as against instability and dissolution caused by moisture, the water content is to be kept as low as possible. Depending on the kind of active substance (hydrophilic, hydrophobic), the water content may be up to 10% by wt. The water content can change after the production of the molded article by freeze drying during storage; as a rule, it increases. Preferably, the water content of the molded article after production is maximally 10% by wt., preferably less than 5% by wt., more preferably less than 1.5% by wt.

A particularly preferred molded article comprises:
≧50% by wt. of one or more active substances, in particular acid active substances, such as ascorbic acid (vitamin C) or its derivatives such as ascorbyl glucoside, or salicylic acid or its derivatives, such as acetylsalicylic acid (ASS)
≦25% by wt., more preferably ≦10% by wt. of one or more scaffold-forming agents, in particular polysaccharides, such as sodium alginate, in particular calcium-free sodium alginate, or cationic scaffold-forming agents, such as chitosan and/or cationized starch or modified scaffold-forming agents, such as carboxymethylcellulose, in particular sodium carboxymethylcellulose or cationized carboxymethylcellulose, as well as mixtures thereof
≦20% by wt. of one or more auxiliary substances, such as, in particular, fats and oils, such as, e.g. neutral oil or triglycerides,
≦10% by wt. of a coating with a film-forming agent, in particular a synthetic hydrophilic polymer, such as vinylpyrrolidone/vinylacetate copolymer (e.g. Kollidon VA64®), and
up to 10% by wt., preferably up to 5% by wt., more preferably less than 1% by wt. of water,
   provided that the molded article decomposes completely and dissolves within ≦30 seconds, preferably ≦20, more preferably ≦10, still more preferably ≦5 seconds when liquid is added, preferably without any mechanical influence.

Preferably, the molded article according to the invention, such as, for example, that of the above-mentioned composition, comprising at least 50% by wt. of one or more active substances, and ≦25% by wt. or ≦10% by wt, of one or more scaffold-forming agents, as well as optionally one or more auxiliary substances, as well as a coating with a film-forming agent, has
a density of 0.005 g/cm³ to 0.8 g/cm³ preferably 0.01 g/cm³ to 0.8 g/cm³,
a volume of 0.1 cm³ to 6 cm³, preferably 0.6 cm³ to 6 cm³,
a diameter (maximum distance between two points of the molded article) of at least 6 mm, and/or
preferably, a spherical configuration, particularly preferably the shape of a sphere.

The molded articles according to the invention constitute porous molded articles with a homogeneous distribution of the ingredients in the core and a thin outer coating with a film-forming agent.

In this case, other ingredients can optionally added to the coating, such as, for example, colorants, or other active or auxiliary substances, such as, for example, inorganic salts, catalysts, such as, for example, enzymes, buffer substances, hygroscopically relevant substances, antimicrobial substances, such as, for example, colloidal silver or silver compounds. It is conceivable in this case, in particular, to provide, by means of the coating and the physical/chemical inhomogeneity of the entire molded article, a system which intentionally makes use of this inhomogeneity or spatial separation in two phases, in order to keep two phases separate until the dissolution and thus complete homogenization of the entire material of the molded article occurs. This can be exploited, for example, in order to obtain a spatial separation of two chemical reactants in the inner core and in the outer coating, which prevents a reaction in the dry, storable state, and which leads to a mobilization and thus reaction of the two hitherto separate immobilized reactants only upon activation by hydration or dissolution of one system. In this way, for example, more stable active substance derivatives can be provided in the core of the composition, and the chemical conversion agents, which release the active but more unstable active substances from these derivatives or precursors, in the outer shell. A premature reaction is prevented by the spatial separation and immobilization in separate layers. It is also possible to provide molded articles that are attractive with regard to their coloring by adding colorants to the shell layer. By concentrating the colorants, for example, in an outer thin coating layer, a significantly more pronounced coloring can be achieved with little colorant, than if the same amount of colorant is homogeneously present in the entire molded article composition. This is advantageous for achieving a good coloring with low colorant contents, without undesired coloring residues remaining on the skin during external application by colorant content that are too high.

The molded articles according to the invention, such as, for example, those as mentioned above, are preferably dissolved with an aqueous liquid/activator solution, which comprises:
at least 70% by wt. of water,
at least 5% by wt. of polyalcohols,
up to 10% by wt. of one or more active substances, such as, in particular, those from the group of the cosmetic active substances
up to 20% by wt. of one or more auxiliary substances, such as in particular those from the group of cosmetic oils, such as in particular caprylic/capric acid triglycerides or jojoba oil, and which has a pH value of 5-7 and, furthermore, a content of earth-alkaline ions, such as, in particular, calcium and/or magnesium ions of less than 1% by wt.

The dissolution rate of the molded articles according to the invention, measured in accordance with a method for measuring the “disintegration time of tablets and capsules” with a testing apparatus according to PharmEU, is less than 30 seconds, preferably less than 20 seconds, more preferably less than 10 seconds, particularly preferably less than 5 seconds.
Moreover, the subject matter of the invention is a method for the production of a freeze-dried coated molded article, characterized in that coating is carried out on the frozen molded article, and the coated frozen molded article is then freeze-dried.
In particular, the subject matter of the invention is a method comprising the following steps:
(a) preparing an aqueous solution or suspension of one or more active substances, optionally of one or more scaffold-forming agents, as well as, optionally, one or more auxiliary substances,
(b) pouring the mixture into a mold
(c) freezing the mixture in the mold, obtaining frozen molded articles
(d) removing the frozen molded articles from the mold and, optionally, rounding the frozen molded articles
(e) spraying the frozen molded articles with a coating composition, comprising at least one film-forming agent and at least one solvent and/or dispersing agent
(f) freeze-drying the frozen molded articles coated with the film-forming agent, while the freeze-dried molded article is formed.

Expediently, production is carried out by first preparing an aqueous solution of the active substances and optionally mixing in a solution of the scaffold-forming agent with stirring. Subsequently, further auxiliary substances are optionally added to the mixture and mixed.

The amount of the solids contained in the solution or suspension, such as scaffold-forming agents, active substances and auxiliary substances is an important influence on the density (weight of the molded article relative to the volume of the geometrical form of the molded article) of the molded article obtained. The density in turn is an important quantity for the porosity of the molded article, and thus for the dissolution rate of the molded article when moistened with water or a solution of active and/or auxiliary substances. The porous structure of freeze-dried molded articles is an essential basis for rapid solubility because an intimate exchange between the aqueous phase and the solid molded article can occur during the rehydration process due to the large surface area in the porous material. The higher the concentration of the active substances, of the scaffold-forming agent as well as of, optionally, the auxiliary substances in the solution, the higher the density becomes, and thus, the lower the degree of porosity of the molded article and vice versa. However, the degree of porosity of the molded articles does not depend on the material density alone. Rather, material porosity is substantially a function of two parameters, the material density and the ice-crystal size. High solid contents in the aqueous suspension increase material density in the freeze-dried final product and reduce the contact surface between the rehydration agent/solid. Large freezing gradients lead to small ice crystals, which lead to large internal material surfaces, which in turn promotes rehydration. Thus, small material densities and small ice crystals are advantageous for quick moisturization and dissolution of the freeze-dried molded articles.

Since inhomogeneous materials are being obtained by coating the frozen molded articles and because the porous core obtainable within the molded articles is shielded towards the outside by a film-forming layer, the configuration of the film coating and its behavior towards the media used for dissolution is of fundamental importance for the dissolution of the coated molded articles. Porous film coatings, in particular, enable a transport of the liquids into the highly porous core, and thus a rapid dissolution of the molded articles. Because dissolution media on an aqueous basis are preferably used for cosmetic and pharmaceutical application, it is important that the coating be selected from the group of hydrophilic film-forming agents in order to avoid problems in moistening and dissolving the coated molded articles. Moreover, the moisture permeability of the film-forming agent in the freeze-drying process is relevant for the above-mentioned reasons, because the water vapor has to escape through the film layer from the molded article during sublimation.

With regard to density/degree of porosity and dissolution rate, respectively, the formulation of the recipe and the production of the molded articles according to the invention is arranged such that the densities of the molded articles that can be obtained therewith are expediently about 0.01 g/cm³ to 0.8 g/cm³, preferably about 0.015 g/cm³ to 0.5 g/cm³, preferably about 0.02 g/cm³ to 0.3 g/cm³. The term density as it is presently used denotes the weight of the molded article relative to the volume of the exterior geometric shape of the composition.

The weight of the individual molded articles is of course dependent upon their size. Generally, the weight of the individual molded articles is about 10 to 300 mg, preferably 20 to 200 mg. For example, spheres with a diameter of 11 mm have a weight in the range from, preferably, 20 to 160 mg, more preferably 30 to 150 mg. Other preferred ranges are calculated correspondingly for spheres having other diameters.

The production of the solution that is to be subjected to freeze drying is preferably carried out by first preparing an aqueous solution of the active substances. Into which a solution of one or more scaffold-forming agents is optionally mixed Subsequently, further auxiliary substances are optionally admixed to the mixture. If oil-soluble active substances are used, they are preferably dissolved in oils that are optionally used as auxiliary substances (in particular squalane and triglycerides) and then added to the aqueous solution of the active substances or of the scaffold-forming agent. This production method is advantageous in that stable solutions or suspensions form in a short time. No emulsifiers or only small amounts of surfactants, such as, for example tensides or wetting agents, are required, and no phase separation of the solution or suspension occurs during processing if oil-soluble or oily auxiliary or active substances are used. Preferably, however, water-soluble active substances are used.

The solution or suspension thus produced is then poured into molds which have cavities of the desired geometric shapes corresponding to the molded articles. The mold preferably consists of rubber, silicone rubber, vulcanized rubber (rubber) etc. Rubber molds are preferred. The mold materials may optionally be coated. The cavities of the molded articles into which the solution is poured generally have the shape of the desired molded article. That is, the volume of the cavity substantially corresponds to the volume of the molded article that is obtained later.

Since the volume of the solutions or suspensions filled in the cavities increases during freezing (difference in density between water and ice), the cavities are generally not filled completely. In this way, completely symmetrical molded articles are obtained. For example, this is not possible according to the method of dripping into cryogenic solutions (such as in liquid nitrogen), because in that case, unsymmetrical temperature distribution occurs, so that greater or lesser deviations from a regular form result every time. Such irregularly shaped molded articles, however, are not desired especially in the area of cosmetic final products. As a rule, this means that molded articles produced according to the dripping method require mechanical post-processing, which is not necessary according to the method as it is used according to the invention. In the case of molded articles produced with the dripping method, such post-processing becomes ever more necessary with an increasing volume of the molded article, because significant external irregularities occur in this method, which become more apparent in the case of large molded articles.

After the solution has been filled into the cavities of the mold, the solution or suspension is frozen. Cooling or freezing the solution can take place, as such, in any way, such as, for example, by blowing with cold air, cooling by applying on a plate through which cooling brine flows, or also dipping the molds into liquid gases, such as dipping into liquid nitrogen. The cooling rate in the process has an effect upon the size of the ice crystals formed. They in turn have an effect upon the pore size distribution in the inner core of the molded article formed. If few large crystals are formed, then the molded article has few large pores, if many small crystals are formed, than the molded article has many small pores. The higher the cooling-off rate of the solution or suspension, the smaller the crystals become. In this case, a freezing geometry in which the molded articles are frozen simultaneously from all sides at least <−20° C. are preferred.

The freezing temperature required depends, among other things, on how large the freezing-point lowering action of the active agents or auxiliary substances contained in the solution or suspension is. Expediently, the temperature is below the freezing point of water, down to the temperature of liquid nitrogen (−196° C.). Preferably, the freezing temperature is about −20° C. to −80° C., particularly preferably −30° C. to 50° C. After the solution or suspension has frozen, the molded articles are removed from the mold and rounded, if necessary. Then, the coating is applied by spraying the frozen molded articles with a suitable coating composition, comprising at least one film-forming agent and at least one solvent and/or dispersing agent. Preferably, such a solvent and/or dispersing agent is a water-alcohol mixture.

The coating composition preferably is a composition of ≧15% by wt. alcohol, ≦70% by wt. water and ≧5% by wt. film-forming agent, in each case based on the total quantity of the coating composition.

Optionally, the aqueous solution of the hydrophilic film-forming agent can contain freezing-point lowering substances, such as, for example, monohydric or polyhydric alcohols or salts. A premature ice formation during the spraying process is prevented by the freezing-point lowering substances. Moreover, the outside of the frozen molded article is thawed for a short time by means of these additives, and the film-forming polymer can form a stable film that is firmly connected with the frozen molded article. Preferably, freezing-point lowering substances are selected which can be removed again from the molded article within the context of freeze drying. The monohydric alcohols methanol and ethanol, in particular, are included amongst them. It is also possible to roll or dip the frozen molded articles in the solution of the film-forming agent. In this case, the solution of the film-forming agent in the solvent and/or dispersing agent, preferably in a water-alcohol mixture, preferably has a composition of at least 5% by wt. alcohol, at least 50% by wt. water and at least 10% by wt. film-forming agent, in each case based on the total quantity of the solution. In principle, it is possible to dissolve the film-forming agent in all alcohols or solvents that can be mixed with water. However, the use of a water-ethanol mixture is particularly preferred.

Optionally, other substances such as colorants, active or auxiliary substances that are soluble in the mixture of water, alcohol and film-forming agent can be added thereto, as well as suitable substances for the chemical conversion of active-substance derivatives and/or precursors.

The molded articles thus coated are then subjected to the freeze-drying process. Freeze drying can take place in a manner known per se, by means of generally known freeze-drying processes such as also described, for example, in DE 4328329 C2, in DE 4028622 C2 or in DE 10350654 A1.

The invention in particular includes the following preferred embodiments:
1. Freeze-dried molded article comprising: at least one or more active substances and optionally one or more scaffold-forming agents, optionally one or more auxiliary substances, as well as a surface coating comprising at least one film-forming agent,
2. Freeze-dried molded article according to embodiment 1, comprising a content of active substances of ≧50% by wt., preferably ≧75% by wt., more preferably ≧80% by wt., still more preferably ≧90% by wt., based on the total composition of the freeze-dried coated molded article.
3. Freeze-dried molded article according to any one of the embodiments 1 or 2, comprising a total content of active and auxiliary substances of a ≧50% by wt., preferably ≧75% by wt., more preferably ≧80% by wt., still more preferably ≧90% by wt., based on the total composition of the freeze-dried, coated molded articles.
4. Freeze-dried molded article according to any one of the embodiments 1 to 3, with a weight ratio of active substances to auxiliary substances of 10:1 to 100:1, in each case relative to the total amount of the active substances in % by wt. in relation to the total amount of the auxiliary substances in % by wt.
5. Freeze-dried molded article according to any one of the embodiments 1 to 4, wherein a 1% by wt. solution or suspension of the active substance in water, at 20° C., has a pH value <7.
6. Freeze-dried molded article according to any one of the embodiments 1 to 4, wherein the active substance is selected from the group of acid active substances having a pka value ≦7 at 25° C.
7. Freeze-dried molded article according to any one of the embodiments 1 to 6, wherein the active substance is selected from the group consisting of ascorbic acid and its derivatives, salicylic acid and its derivatives, in particular acetylsalicylic acid, clofibric acid, ibuprofen, gemfibrozil, fenoprofen, naproxen, ketoprofen, indomethacin, bezafibrate, tolfenamine acid, diclofenac, meclofenamine acid, paracetamol, acitretine, acrivastine, azelaic acid, cromolyn, ethacrynic acid, furosemide, penicillin and derivatives thereof, vitamin A and derivatives thereof, risedronic acid and derivatives thereof, lipoic acid and ursodiol.
8. Freeze-dried molded article according to embodiment 7, wherein at least one acid active substance is selected from the group of ascorbic acid and its derivatives, or from the group of acetylsalicylic acid and its derivatives.
9. Freeze-dried molded article according to any one of the embodiments 1 to 8, which, based on the total composition of the freeze-dried coated molded article, contains ≧50% by wt. of an active substance from the group of ascorbic acid and its derivatives.
10. Freeze-dried molded article according to any one of the embodiments 1 to 8, which, based on the total composition of the freeze-dried coated molded article, contains ≧50% by wt. of an active substance from the group of salicylic acid and its derivatives, preferably from the group of acetylsalicylic acid and its derivatives.
11. Freeze-dried molded article according to any one of the embodiments 1 to 10, with a content of scaffold-forming agents of ≦25% by wt., preferably ≦10% by wt., more preferably ≦5% by wt, in each case based on the total composition of the freeze-dried coated molded article.
12. Freeze-dried molded article according to any one of the embodiments 1 to 11, in which no scaffold-forming agents are contained.
13. Freeze-dried molded article according to any one of the embodiments 1 to 12, wherein the scaffold-forming agent is selected from the group of the hydrocolloids, preferably from the group of polysaccharides, polyaminosaccharides, glucosaminoglycanes and/or synthetic polymers or mixtures thereof.
14. Freeze-dried molded article according to embodiment 13, wherein the scaffold-forming agent is an alginate, preferably a sodium alginate, carboxymethylcellulose, canonically modified carboxymethylcellulose, canonically modified starch or chitosan or a mixture thereof.
15. Freeze-dried molded article according to any one of the embodiments 1 to 14, wherein scaffold-forming agents from the group of proteins are excepted.
16. Freeze-dried molded article according to any one of the embodiments 1 to 15, wherein the content of the coating with a film-forming agent is ≦10% by wt., preferably ≦7% by wt., more preferably ≦5% by wt., in each case based on the total composition of the freeze-dried coated molded article.
17. Freeze-dried molded article according to any one of the embodiments 1 to 16, wherein the film-forming agent for the coating is selected from the group of hydrophilic polymers.
18. Freeze-dried molded article according to embodiment 17, wherein the film-forming agent for the coating is selected from the group of synthetic polymers, preferably from the group of vinylpyrrolidone/vinylacetate copolymers.
19. Freeze-dried molded article according to any one of the embodiments 1 to 18, characterized in that it decomposes completely within ≦30 seconds, preferably ≦20 seconds, more preferably ≦10 seconds, still more preferably ≦5 seconds when liquid is added.
20. Freeze-dried molded article according to any one of the embodiments 1 to 19, which has a volume of 0.1 cm³ to 6 cm³, a density of 0.01 g/cm³ to 0.8 g/cm³ and/or the geometrical shape of a sphere with a diameter of at least 6 mm.
21. Method for the production of a freeze-dried coated molded article, which is characterized in that coating is carried out on the frozen molded article, and the coated frozen molded article is then freeze-dried.
22. Method for producing a freeze-dried molded article, comprising the following steps
   a. preparing an aqueous solution or suspension of one or more active substances, optionally of one or more scaffold-forming agents, as well as, optionally, one or more auxiliary substances,
   b. pouring the mixture into a mold
   c. freezing the mixture in the mold, obtaining frozen molded articles
   d. removing the frozen molded articles from the mold and, optionally, rounding
   e. spraying the frozen molded articles with a coating composition, comprising at least one film-forming agent and at least one solvent and/or dispersing agent
   f. freeze-drying the frozen molded articles coated with the film-forming agent, while the freeze-dried molded article is formed.
23. Method according to any one of the embodiments 21 or 22, wherein a film-forming agent is used for the coating which is selected from the group of hydrophilic polymers, preferably from the group of synthetic polymers, particularly preferably from the group of vinylpyrrolidone/vinylacetate copolymers.
24. Method according to embodiment 22 or 23, wherein the solvent and/or dispersing agent is a water-alcohol mixture.
25. Method according to any one of the embodiments 22 to 24, wherein the coating composition is a composition of ≧15% by wt. alcohol, ≧70% by wt. water and ≧5% by wt. film-forming agent, in each case based on the total quantity of the coating composition.
26. Freeze-dried molded article that can be obtained in accordance with the method according to any of the embodiments 21 to 25.
27. Use of the freeze-dried molded article according to any one of the embodiments 1 to 20 or 26 as a cosmetic agent.
28. Use of the freeze-dried molded article according to any one of the embodiments 1 to 20 or 26 as a pharmaceutical agent.
29. Use according to embodiment 27 or 28, wherein the application takes place externally.
30. Use according to any one of the embodiments 27 to 29, wherein the freeze-dried molded article is moistened with water or an aqueous solution of one or more active substances and/or, optionally, auxiliary substances and decomposes within ≦30 seconds and is then applied onto the skin or hair.
31. Use of the freeze-dried molded article according to any one of the embodiments 1 to 20 or 26 for the oral or peroral application of active substances.
32. Kit-of-parts combination, comprising at least one freeze-dried molded article according to any one of the embodiments 1 to 20 or 26, as well as at least one aqueous solution comprising one or more active substances and/or, optionally, one or more auxiliary substances, in a combined spatial arrangement.
33. Use of the kit-of-parts combination according to embodiment 32 as a cosmetic agent.
34. Use of the kit-of-parts combination according to embodiment 32 as a therapeutic agent.
35. Use according to any one of the embodiments 27 to 31 and 33 to 34, which is carried out directly by the end user.

The invention is illustrated in more detail by the following examples.

### EXAMPLES

### Example 1

### Production of a Carboxymethylcellulose/Ascorbic Acid Sphere with Subsequent Coating

0.5 g carboxymethylcellulose

16.0 g ascorbic acid

83.5 g water

0.5 g carboxymethylcellulose are added to 83.5 g water with stirring and stirred until the carboxymethylcellulose has dissolved completely and homogeneously. 16.0 g ascorbic acid are then added with stirring, the mixture, which has a pH value of 3.0, is kept at a temperature of 0-10° C. in the process. The homogeneous (degassed) mixture is poured into molds, frozen through with blowing of cold air, taken out of the mold and optionally post-processed mechanically. Frozen spheres with a diameter of approximately 11 mm diameter are obtained, which can optionally be stored in the frozen state at temperatures below −20° C.
In a cold-storage room at temperatures <−10° C., the frozen molded articles are sprayed homogeneously, with shaking, with a solution of:
80 g ethanol
10 g RO water (reverse osmosis water)

### 10 g Kollidon VA-64

until all spheres are uniformly coated with the coating agent. The excess ethanol is allowed to evaporate with shaking. The coated, deep-frozen spheres are then subjected to freeze drying. Mechanically stable freeze-dried molded articles coated with Kollidon VA-64 are obtained.
The quantity of coating agent, and thus, the mechanical stability of the freeze-dried molded article, can easily be controlled via the quantity of sprayed-on coating agent.
The dissolution rate of the freeze-dried coated molded articles, measured in accordance with a method for measuring the “disintegration time of tablets and capsules” with a testing apparatus according to PharmEU, is less than 10 seconds.

### Example 2

### Production of a Chitosan/Acetylsalicylic Acid Sphere with Subsequent Coating

0.2 g chitosan

16.0 g acetylsalicylic acid

83.8 g water

0.2 g chitosan is added to 83.8 g water with stirring, some diluted hydrochloric acid is added, and stirring is continued until the chitosan has dissolved completely. 16.0 g acetylsalicylic acid are then dispersed into the chitosan solution with stirring, the mixture, which has a pH value of ≦3.0, is kept at a temperature of 0-10° C. in the process. The homogeneous (degassed) mixture is poured into molds, frozen through with blowing of cold air, taken out of the mold and optionally post-processed mechanically. Frozen spheres with a diameter of approximately 11 mm diameter are obtained, which can optionally be stored in the frozen state at temperatures below −20° C. In a cold-storage room at temperatures <−10° C., the frozen molded articles are sprayed homogeneously, with shaking and blowing of cold air, with a solution of:
85 g ethanol

### 15 g Kollidon VA-64

until all spheres are uniformly coated with the coating agent. The excess ethanol is allowed to evaporate with shaking and blowing with cold air. The coated, deep-frozen spheres are then subjected to freeze drying. Mechanically stable freeze-dried molded articles coated with Kollidon VA-64 are obtained.
The quantity of coating agent, and thus, the mechanical stability of the freeze-dried molded article, can easily be controlled via the quantity of sprayed-on coating agent.
The dissolution rate of the freeze-dried coated molded articles, measured in accordance with a method for measuring the “disintegration time of tablets and capsules” with a testing apparatus according to PharmEU, is less than 10 seconds.

### Example 3

### Production of a Ascorbic Acid Sphere with Subsequent Coating

16.0 g ascorbic acid

84.0 g water

16.0 g ascorbic acid are dissolved in 84.0 g water. The pH value of the solution is ≦pH 3.0, and the solution is kept at a temperature of 0-10° C. The homogeneous (degassed) solution is poured into molds, frozen through with blowing of cold air, taken out of the mold and optionally post-processed mechanically. Frozen ascorbic acid spheres with a diameter of approximately 11 mm diameter are obtained, which can optionally be stored in the frozen state at temperatures below −20° C.
In a cold-storage room at temperatures <−10° C., the frozen molded articles are sprayed homogeneously, with shaking, with a solution of:
90 g ethanol

### 10 g Kollidon VA-64

until all spheres are uniformly coated with the coating agent. The excess ethanol is allowed to evaporate with shaking. The coated, deep-frozen spheres are then subjected to freeze drying. Mechanically stable freeze-dried molded articles of ascorbic acid coated with Kollidon VA-64 are obtained.
The quantity of coating agent, and thus, the mechanical stability of the freeze-dried molded article, can easily be controlled via the quantity of sprayed-on coating agent.
The dissolution rate of the freeze-dried coated molded articles, measured in accordance with a method for measuring the “disintegration time of tablets and capsules” with a testing apparatus according to PharmEU, is less than 5 seconds.

### Example 4

### Production of a Cationized Starch/Ascorbic Acid Sphere with Subsequent Coating

2.0 g cationized starch

1.0 g jojoba oil

16.0 g ascorbic acid

81.0 g water

2.0 g cationized starch is added with stirring into 81.0 g water, and stirring is continued until the starch has dissolved completely and homogenously. 16.0 g ascorbic acid and 1.0 g jojoba oil are then added with stirring, the mixture, which has a pH value of ≦3.0, is kept at a temperature of 0-10° C. in the process. The homogeneous (degassed) mixture is poured into molds, frozen through with blowing of cold air, taken out of the mold and optionally post-processed mechanically. Frozen spheres with a diameter of approximately 11 mm diameter are obtained, which can optionally be stored in the frozen state at temperatures below −20° C.
In a cold-storage room at temperatures <−10° C., the frozen molded articles are sprayed homogeneously, with shaking, with a solution of:
80 g ethanol
10 g RO water

### 10 g Kollidon VA-64

until all spheres are uniformly coated with the coating agent. The excess ethanol is allowed to evaporate with shaking. The coated, deep-frozen spheres are then subjected to freeze drying. Mechanically stable freeze-dried molded articles coated with Kollidon VA-64 are obtained.
The quantity of coating agent, and thus, the mechanical stability of the freeze-dried molded article, can easily be controlled via the quantity of sprayed-on coating agent.
The dissolution rate of the freeze-dried coated molded articles, measured in accordance with a method for measuring the “disintegration time of tablets and capsules” with a testing apparatus according to PharmEU, is less than 10 seconds.

1. Freeze-dried molded article comprising at least one or more active substances and optionally one or more scaffold-forming agents, optionally one or more auxiliary substances, as well as a coating comprising at least one film-forming agent.

2. Freeze-dried molded article according to claim 1, comprising a content of active substances of ≧50% by wt, preferably ≧75% by wt., more preferably ≧80% by wt., still more preferably ≧90% by wt., based on the total composition of the freeze-dried coated molded article.

3. Freeze-dried molded article according to claim 1, wherein the active substance is selected from the group of acid active substances having a pKa value ≦7 at 25° C.

4. Freeze-dried molded article according to claim 1, which, based on the total composition of the freeze-dried coated molded article, contains ≧50% by wt. of an active substance from the group of ascorbic acid and its derivatives or the group of salicylic acid and its derivatives, preferably from the group of acetylsalicylic acid and its derivatives.

5. Freeze-dried molded article according to claim 1, with a content of scaffold-forming agents of ≦10% by wt., more preferably ≦5% by wt., based on the total composition of the freeze-dried coated molded article.

6. Freeze-dried molded article according to claim 1, in which no scaffold-forming agents are contained.

7. Freeze-dried molded article according to claim 1, wherein the scaffold-forming agent is selected from the group of the hydrocolloids, preferably from the group of polysaccharides, polyaminosaccharides, glucosaminoglycanes and/or synthetic polymers or mixtures thereof.

8. Freeze-dried molded article according to claim 7, wherein the scaffold-forming agent is an alginate, preferably a sodium alginate, carboxymethylcellulose, or chitosan or a mixture thereof.

9. Freeze-dried molded article according to claim 1, wherein the content of the coating with a film-forming agent is ≦10% by wt., preferably ≦7% by wt., more preferably ≦5% by wt., in each case based on the total composition of the freeze-dried coated molded article.

10. Freeze-dried molded article according to claim 1, wherein the film-forming agent for the coating is selected from the group of hydrophilic polymers, preferably from the group of synthetic polymers, preferably from the group of vinylpyrrolidone/vinylacetate copolymers.

11. Freeze-dried molded article according to claim 1, which decomposes completely within ≦30 seconds, preferably ≦20 seconds, more preferably ≦10 seconds, still more preferably ≦5 seconds when liquid is added.

12. Method for the production of a freeze-dried coated molded article, wherein coating is carried out on the frozen molded article, and the coated frozen molded article is then freeze-dried.

13. Method for producing a freeze-dried molded article, comprising the following steps
(a) preparing an aqueous solution or suspension of one or more active substances, optionally of one or more scaffold-forming agents, as well as, optionally, one or more auxiliary substances,
(b) pouring the mixture into a mold
(c) freezing the mixture in the mold, obtaining frozen molded articles
(d) removing the frozen molded articles from the mold and, optionally, rounding
(e) spraying the frozen molded articles with a coating composition, comprising at least one film-forming agent and at least one solvent and/or dispersing agent
(f) freeze-drying the frozen molded articles coated with the film-forming agent, while the freeze-dried molded article is formed.

14. Use of the freeze-dried molded article according to claim 1 as a cosmetic agent or as a pharmaceutical agent.

15. Kit-of-parts combination, comprising at least one freeze-dried molded article according to claim 1, as well as at least one aqueous solution comprising one or more active substances and/or, optionally, one or more auxiliary substances, in a combined spatial arrangement.

16. Freeze-dried molded article according to claim 2, wherein the active substance is selected from the group of acid active substances having a pKa values ≦7 at 25° C.

17. Freeze-dried molded article according to claim 16, which, based on the total composition of the freeze-dried coated molded article, contains ≧50% by wt. of an active substance from the group of ascorbic acid and its derivatives or the group of salicylic acid and its derivatives, preferably from the group of acetylsalicylic acid and its derivatives.

18. Freeze-dried molded article according to claim 3, which, based on the total composition of the freeze-dried coated molded article, contains ≧50% by wt. of an active substance from the group of ascorbic acid and its derivatives or the group of salicylic acid and its derivatives, preferably from the group of acetylsalicylic acid and its derivatives.

19. Freeze-dried molded article according to claim 2, which, based on the total composition of the freeze-dried coated molded article, contains ≧50% by wt. of an active substance from the group of ascorbic acid and its derivatives or the group of salicylic acid and its derivatives, preferably from the group of acetylsalicylic acid and its derivatives.

20. Freeze-dried molded article according to claim 17, with a content of scaffold-forming agents of ≦10% by wt., more preferably ≦5% by wt., based on the total composition of the freeze-dried coated molded article.
